(19) United States
(12) Patent Application Publication
(10) Pub. No.: US 2007/0083250 A1
(43) Pub. Date: Apr. 12, 2007

(54) Healthful patch

(76) Inventor: Yu-Ting Tu, Tainan City (TW)

Correspondence Address: Yu-Ting Tu, P.O. Box 90, Tainan City, 70499, TW

(21) Appl. No.: 11/246,055

(22) Filed: Oct. 11, 2005

(51) Int. Cl. A61F 7/00

(52) U.S. Cl. 607/112; 607/114

(57) ABSTRACT

A healthful patch includes an airy cloth and a layer of silicon gel mixed with far-infrared energy powder coated on a lower surface of the airy cloth. The layer of the silicon gel is provided with many tiny air holes. The healthful patch can be covered on an aching or sore spot or section of a human body to let the far-infrared energy functioning to give medicating effect to the aching or sore spot or section for reinforcing blood circulation and metabolism, relieving aching or sore, getting rid of fatigues, activating cells, and giving healthful effect to human bodies.

### BACKGROUND OF THE INVENTION

1. Field of the Invention

This invention relates to a healthful patch, particularly to one provided with a layer of silicon gel mixed with far-infrared energy powder coated on a lower surface of an airy cloth, and the layer of silicon gel provided with many air holes. When the healthful patch (es) is (are) covered on a body section suffering aching pains, with the layer of silicon gel directly contacting the skin, the far infrared energy having medicating function can reinforce blood circulation, step up metabolism of the body of a user, reduce pains, assuage fatigues, activating cells and contribute to the health of the user.

2. Description of the Prior Art

The female generally desire full and plump breasts, so they are eager to go to doctors for receiving plastic surgeries of their breasts. However, the plastic surgery involves some risk, not necessarily making them plump. So a majority of women may resort to food helpful to get them plump or to massage or other ways. But these ways have effects quite limited, some may often use brassieres under their clothes for enlarging breasts in order to show off them. Brassieres are made of various materials, having an airy condition if made of cotton cloth but liable to disfigure after washing. Those made of a plastic film bag filled with liquid do not have an airy condition, easily to cause sweating in summer and let the skin apt to grow measles and have allergy to appear, Moreover, common conventional brassieres do not possess any medicating function to help health to a human body. Not few females may have pains in their lower abdomen when menses come, and be forced to take pain killers for assuaging them, but pain killers may not so good for health to the body. So some may use a heat compress bag to quell the pains of the lower abdomen, with its effect quite limited. In addition, pain-assuaging patches may be used for the same purpose, discarded after use, forming some waste.

### SUMMARY OF THE INVENTION

The main purpose of the invention is to offer a healthful patch possessing good ventilating condition and far infrared energy to supply medicating function for reinforcing blood circulation and metabolism, removing fatigue, lessening aching pains, activating cells, with repeating usability.

The feature of the invention is an airy cloth and a layer of silicon gel mixed with far-infrared energy powder coated on a lower surface of the airy cloth and provided with many tiny air holes. Then the health patch in the invention can be covered directly on a body section with aching pains or the like, and as the far-infrared energy possesses medicating function to a human body for reinforcing blood circulation and metabolism, assuaging aching pains, getting rid of fatigue, activating cells, and thus very helpful to a humane body.

### BRIEF DESCRIPTION OF DRAWINGS

This invention will be better understood by referring to the accompanying drawings, wherein:

FIG. 1 is a perspective view of a healthful patch in the present invention, showing it used as a breast patch;

FIG. 2 is a magnified cross-sectional view of the healthful patch in the present invention;

FIG. 3 is a perspective view of the healthful patch in the present invention, showing it used as an abdomen patch;

FIG. 4 is a perspective view of the healthful patch formed rectangular in the present invention; and, FIG. 5 is a perspective view of the healthful patches in the present invention, showing them practically used on a human body.

### DETAILED DESCRIPTION OF THE PREFERRED EMBODIMENT

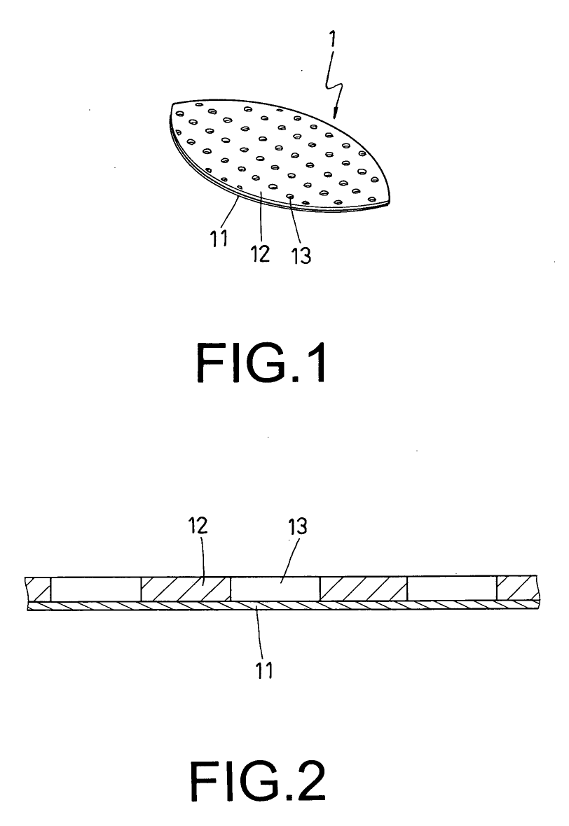

A preferred embodiment of a healthful patch in the present invention, as shown in FIGS. 1 and 2, includes a cloth 11 of a ventilating property, and a layer of silicon gel 12 coated on a lower surface of the cloth 11. The cloth 11 may be nonwoven cloth, knitted cloth or common woven cloth. The silicon gel 12 is made of silicon gel mixed with far-infrared energy powder, and this mixture is poured in a mold and heated to be coated hardened on a lower surface of the cloth 11, and the layer of the silicon gel 12 have many tiny air holes 13. The healthful patch shown in FIGS. 1 and 2 is made to be used to cover on a breast.

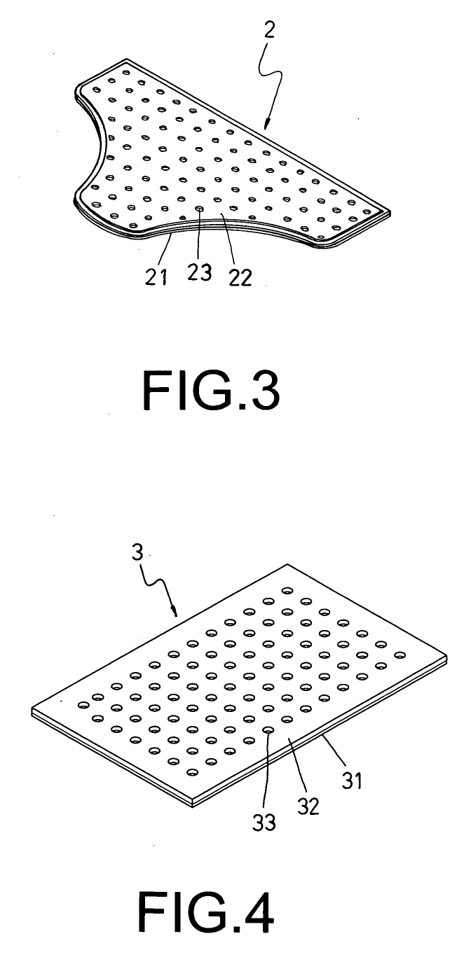

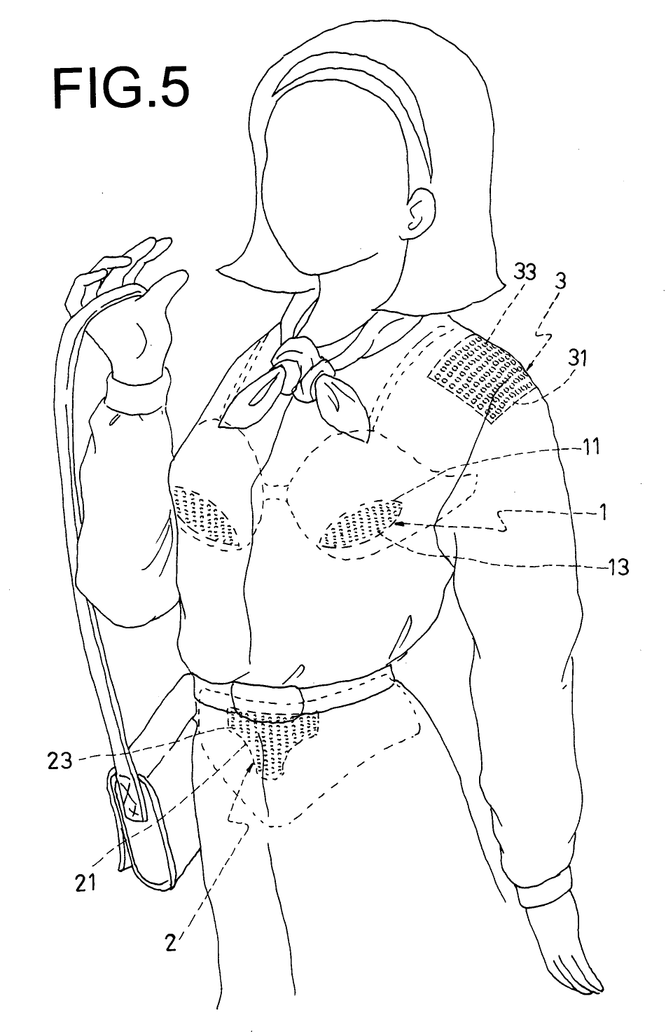

The healthful patch in the invention may also be formed as an abdomen patch 2 as shown in FIG. 3, or as a rectangular patch 3 shown in FIG. 4, or any other shape for different sections of a human body. In the abdomen patch 2 is used an airy cloth 21 and a layer of silicon gel 22 mixed with far-infrared energy powder coated on a lower surface of the airy cloth 21, and in the rectangular patch 3 is used an airy cloth 31 and a layer of silicon gel 32 mixed with far-infrared energy powder coated on a lower surface of the cloth 31.

Next, in using, as shown in FIG. 5, the patch 1, 2 or 3, is covered on an aching spot of a human body or on a section to be treated for healthful purpose, for example, the breast patch 1 is placed on a lower section of a breast, and then a brassiere is worn to keep stable the breast patch 1. In case of the abdomen patch 2, a user places it on the abdomen, and as for the rectangular patch 3, it may be placed on any needed section of a body. Thus, the layer of the silicon gel 12, 22 or 32 contacts directly the skin so that the far-infrared energy mixed therein may sink in the skin system and organs for generating medicating function. As for the breast patch, it may improve the breast system, activate cells, and promote blood circulation and metabolism for rounding the breast. The abdomen patch 2 may reinforce blood circulation and metabolism, assuage fatigue, activate cells, relieving pains of the abdomen, and lessening feminine disorders. In case of the rectangular patch, it can be placed any section of a body for grading up blood circulation and metabolism, activating cells, relieving fatigues, and reducing aching pains or sores. Besides, the layer of the silicon gel 12, 22 or 32 has many airy holes 13, 23 or 33 for ventilation, and a soft property to let a user feel comfortable. Moreover, the patch 1, 2 or 3 can be used repeatedly, helpful to the environment preservation and considerably healthful to human bodies.

While the preferred embodiment of the invention has been described above, it will be recognized and understood that various modifications may be made therein and the appended claims are intended to cover all such modifications that may fall within the spirit and scope of the invention.

What is claimed is:

1. A healthful patch comprising an airy cloth and a layer of silicon gel mixed with far-infrared energy powder coated on a lower surface of said airy cloth, said layer of silicon gel provided with many tiny air holes, said patch covered on an aching spot of a human body with said layer of silicon gel directly contacting a skin of said aching spot, said far-infrared energy powder mixed in said layer of silicon gel possessing medicating function so that the aching spot may receive medicating function from said layer of silicon gel for reinforcing blood circulation and metabolism, relieving aching pains, getting rid of fatigues, activating cells, and playing healthful role to a human body.

2. The healthful patch as claimed in claim 1, wherein said cloth is nonwoven cloth.

3. The healthful patch as claimed in claim 1, wherein said cloth is knitted cloth.

4. The healthful patch as claimed in claim 1, wherein said cloth is plain woven cloth.

5. The healthful patch as claimed in claim 1, wherein said layer of silicon gel is made of silicon gel mixed with far-infrared energy powder, and said mixed material is poured in a mold and heated to be coated hardened on the lower surface of said cloth.

6. The healthful patch as claimed in claim 1, wherein said patch is made to have various sizes and shapes to suit to different spots and sections of a human body.
